(19) United States
(12) Patent Application Publication
(10) Pub. No.: US 2016/0010131 A1
(43) Pub. Date: Jan. 14, 2016

(54) METHOD FOR PRETREATING BIOMASS, AND METHOD FOR PRODUCING SUGAR CONTAINING GLUCOSE AS MAIN COMPONENT

(71) Applicant: JGC CORPORATION, Tokyo (JP)

(72) Inventor: DAISUKE TANEDA, IBARAKI (JP)

(21) Appl. No.: 14/770,805

(22) PCT Filed: Feb. 28, 2013
(86) PCT No.: PCT/JP2013/055579

§ 371 Date: Aug. 26, 2015

(51) Int. Cl. C12P 19/14; D21B 1/36; C12P 19/02; D21B 1/02

(52) U.S. Cl. CPC C12P 19/14; D21B 1/021; D21B 1/36; C12P 19/02; C12P 2201/00

(57) ABSTRACT

A biomass pretreatment method includes a first process performed on the biomass so as to improve contact efficiency between the cellulose and/or hemicellulose and the enzymes that degrade the cellulose and/or hemicellulose; and a second process of removing a substance containing at least an organic acid from the biomass subjected to the first process, the second process including placing the biomass subjected to the first process into a container that has a vent communicating with outside and heating the biomass subjected to the first process at a temperature equal to or lower than a boiling temperature of water by using water while leaving the vent constantly open.

### TECHNICAL FIELD

The present invention relates to a method for pretreating a biomass to be supplied to a method for producing saccharides containing glucose as a main component, the method for producing saccharides including enzymatically degrading cellulose and/or hemicellulose contained in a biomass, and a method for producing saccharides containing glucose as a main component by using the biomass pretreatment method.

### BACKGROUND ART

An example of a method for producing bioethanol from a cellulosic biomass as a raw material is an enzymatic ethanol production technique that involves enzymatically hydrolyzing cellulose and/or hemicellulose. The greatest challenge in putting the enzymatic ethanol production technique in practical use is the reduction of the amount of enzymes used. In order to reduce the amount of enzymes used, a biomass needs to be subjected to a particular treatment to increase the contact efficiency between enzymes and cellulose and/or hemicellulose. This treatment is called a pretreatment.

In order to maximize the contact efficiency between the enzymes and cellulose and/or hemicellulose, lignin must be completely removed from the biomass. For example, it is technologically possible to nearly completely remove lignin by employing a method of dissolving lignin in a highly concentrated alkali or the like; however, this pretreatment method requires a high treatment cost.

Thus, instead of completely removing lignin, a pretreatment method of breaking-down or partially dissolving lignin so as to form spaces in a biomass where enzymes can be brought into contact with cellulose or hemicellulose has been mainly developed. According to this method, the treatment cost is low; however, if the spaces are small or few, the contact efficiency between the enzymes and cellulose or hemicellulose is decreased, resulting in a low saccharide recovery rate. Moreover, organic acids, tar components, and other components that inhibit enzymatic hydrolysis occur as a result of the pretreatment operation, and remain in the biomass. Accordingly, a novel pretreatment technique capable of producing a biomass that can be more easily enzymatically saccharified at a low cost is required.

An example of a biomass pretreatment technique is a technique developed by National Renewable Energy Laboratory (NREL) in USA and called a “hot wash”, in which corn stover (stalks, etc., of corn plant) treated with acids or hot water is used.

The hot wash developed by NREL is a technique of washing away soluble components by washing acid- or hot-water-processed corn stover with hot water or diluted sulfuric acid heated to about 140° C. (for example, refer to PTL 1). Since a pretreated biomass contains components that inhibit enzymatic saccharification, such components are washed away with hot water or diluted sulfuric acid according to this technique.

### REFERENCE LIST

### Patent Literature

PTL 1: U.S. Pat. No. 6,228,177

### SUMMARY OF INVENTION

### Technical Problem

According to the hot wash technique described above that uses hot water or diluted sulfuric acid of 100° C. or higher, the pressure of the hot water or diluted sulfuric acid is equal to or higher than the atmospheric pressure. Thus, washing cone stover with hot water or diluted sulfuric acid of 100° C. or higher must be performed inside a filtering system or the like that can maintain such a pressure, thereby requiring a high facility cost, which may pose a problem.

The present invention has been made under the above-described circumstances and aims to provide a biomass pretreatment method that enables high-yield production of saccharides containing glucose as a main component from cellulose and/or hemicellulose contained in the biomass by using less enzymes, and a method for producing saccharides containing glucose as a main component by using the biomass pretreatment method.

### Solution to Problem

A biomass pretreatment method of the present invention is a method for pretreating a biomass to be supplied to a method for producing saccharides containing glucose as a main component, the method for producing saccharides including degrading cellulose and/or hemicellulose contained in a biomass with enzymes that degrade cellulose and/or hemicellulose. The biomass pretreatment method includes a first process performed on the biomass so as to improve contact efficiency between the cellulose and/or hemicellulose and the enzymes that degrade the cellulose and/or hemicellulose; and a second process of removing a substance containing at least an organic acid from the biomass subjected to the first process, the second process including placing the biomass subjected to the first process into a container that has a vent communicating with outside and heating the biomass subjected to the first process at a temperature equal to or lower than a boiling temperature of water by using water while leaving the vent constantly open.

In the method for pretreating a biomass according to the present invention, the first process is preferably a steam explosion process or a diluted sulfuric acid process.

In the method for pretreating a biomass according to the present invention, the second process preferably involves heating the biomass by using water at 100° C. or lower.

In the method for pretreating a biomass according to the present invention, in the second process, X representing a temperature of water used to heat the biomass and Y representing a length of time the biomass is heated, and X and Y preferably satisfy formula (1) below:

Y≧0.0175X²−3.485X+174 (1)

A method for producing saccharides containing glucose as a main component according to the present invention includes degrading, by using enzymes that degrade cellulose and/or hemicellulose, cellulose and/or hemicellulose contained in a biomass pretreated by the method for pretreating a biomass according to the present invention so as to obtain saccharides containing glucose as a main component.

### Advantageous Effects of Invention

According to the present invention, a substance containing at least an organic acid can be removed from a biomass by performing the second process of placing the biomass subjected to the first process into a container that has a vent communicating with outside and heating the biomass subjected to the first process at a temperature equal to or lower than a boiling temperature of water by using water while leaving the vent constantly open. Thus, when the pretreated biomass is used in enzymatic ethanol production techniques, saccharides containing glucose as a main component can be produced in high yield with less enzymes from cellulose and/or hemicellulose contained in the biomass.

### BRIEF DESCRIPTION OF DRAWINGS

FIG. 1 is a graph showing the relationship between the reaction time of enzymatic degradation and the concentration of glucose produced in Example 1 and Comparative Example 1.

FIG. 2 is a graph showing the relationship between the reaction time of enzymatic degradation and the concentration of glucose produced in Example 2 and Comparative Example 2.

FIG. 3 is a graph showing the relationship between the reaction time of enzymatic degradation and the concentration of glucose produced in Example 3 and Comparative Example 3.

FIG. 4 is a graph showing the relationship between the length of time steam-exploded bagasse is boiled and the concentration of glucose produced in Experimental Example.

### DESCRIPTION OF EMBODIMENTS

Embodiments of a biomass pretreatment method and a method for producing saccharides containing glucose as a main component by using the biomass pretreatment method according to the present invention will now be described.

The embodiments provide specific descriptions that bring better understanding of the gist of the invention and do not limit the present invention unless specifically designated.

### Biomass Pretreatment Method

A biomass pretreatment according to this embodiment is a method for pretreating a biomass to be used in a method for pretreating a biomass to be supplied to a method for producing saccharides containing glucose as a main component, the method for producing saccharides including degrading cellulose and/or hemicellulose contained in a biomass with enzymes that degrade cellulose and/or hemicellulose. The biomass pretreatment method includes a first process performed on the biomass so as to improve contact efficiency between the cellulose and/or hemicellulose and the enzymes that degrade the cellulose and/or hemicellulose; and a second process of removing a substance containing at least an organic acid from the biomass subjected to the first process, the second process including placing the biomass subjected to the first process into a container that has a vent communicating with outside and heating the biomass subjected to the first process at a temperature equal to or lower than a boiling temperature of water by using water while leaving the vent constantly open.

In the biomass pretreatment method of this embodiment, first, a first process is performed on a biomass (wood, grass, or residue of agricultural crops) so as to improve contact efficiency between the cellulose and/or hemicellulose contained in the biomass and the enzymes that degrade cellulose and/or hemicellulose.

Examples of the first process include an alkali process, an organic solvent process, a diluted sulfuric acid process, and a steam explosion process to be performed on a biomass. From the viewpoints of enzymatic saccharification yield and facility cost, a steam explosion process, an alkali process, or a diluted sulfuric acid process is preferably employed.

The alkali process, the organic solvent process, the diluted sulfuric acid process, and the steam explosion process to be conducted on the biomass may be any known processes.

Next, a second process of removing a substance containing at least an organic acid from the biomass subjected to the first process is performed. The second process includes placing the biomass subjected to the first process into a container that has a vent communicating with outside and heating the biomass subjected to the first process at a temperature equal to or lower than a boiling temperature of water by using water while leaving the vent constantly open.

The vent installed onto the container of the biomass has a pipe shape and communicates with the exterior (outside) of the container. A device such as a heat exchanger is provided in the middle of the pipe.

In the second process, the biomass subjected to the first treatment is heated at a temperature equal to or lower than the boiling temperature of water by using water while constantly leaving the vent open, in other words, without controlling the pressure inside the container.

Examples of the process of heating the biomass, which has been subjected to the first treatment, at a temperature equal to or lower than the boiling temperature of water by using water include a process of boiling the biomass in water having a temperature equal to or lower than the boiling temperature of water (for example, 100° C.) and a process of steaming the biomass with steam having a temperature equal to or lower than the boiling temperature.

In the second process, the temperature of water during heating of the biomass is preferably 100° C. or lower. The temperature of water during heating of the biomass is more preferably 80° C. or higher and 100° C. or lower since the efficiency of removing a substance containing at least an organic acid from the biomass is improved (the substance can be removed from the biomass in a short period of time).

In the second process, the length of time the biomass is heated at a temperature equal to or lower than the boiling temperature of water by using water is preferably 1 to 10 minutes and more preferably 3 to 5 minutes.

In the second process, X representing a temperature of water used to heat the biomass and Y representing a length of time the biomass is heated, and X and Y preferably satisfy formula (1) below:

Y≧0.0175X²−3.485X+174 (1)

In other words, once the temperature of water used to heat the biomass in the second process is set to X (° C.), the length of time the biomass is to be heated can be determined to be Y (min) from formula (1).

The substance removed from the biomass by the second process contains at least an organic acid such as acetic acid, and tar components in addition to the organic acid.

In the second process of heating the biomass, which has been subjected to the first process, at a temperature equal to or lower than the boiling temperature of water by using water, not only the components, such as organic acid, that inhibit enzymatic saccharification remaining in the biomass are more thoroughly removed, but also the following effects are obtained. Those effects include an effect of expanding the space between lignin and cellulose and/or between lignin and hemicellulose and thereby increasing the area in which the enzymes contact cellulose, and an effect of degrading and stripping away those components which inhibit enzymatic saccharification and which remain partly un-degraded in the previous process and do not merely adhere to the biomass but are chemically bonded to lignin.

As a result of the second process, the organic acid concentration in the processed solution (solution containing water and the biomass) increases; hence, solid components are washed or neutralization is performed after the completion of the second process so as to recover the biomass.

According to the biomass pretreatment method of this embodiment, a first process is performed on the biomass to improve the contact efficiency between cellulose and/or hemicellulose and enzymes that degrade cellulose and/or hemicellulose, and then in the second process, the biomass subjected to the first process is heated at a temperature equal to or lower than the boiling temperature of water by using water so as to remove a substance that contains at least an organic acid from the biomass subjected to the first process. Thus, when the biomass that has been subjected to this pretreatment is used in enzymatic ethanol production techniques, saccharides containing glucose as a main component can be produced in high yield with less enzymes from cellulose and/or hemicellulose contained in the biomass. Note that while the cost of enzymes is the largest obstacle for production of saccharides containing glucose as a main component, the biomass pretreatment method of this embodiment enables reduction in amount of enzymes used and can cut the cost of producing saccharides containing glucose as a main component. Moreover, since there is no need to use hot water or diluted sulfuric acid of 100° C. or higher as in the related art, no filtering system that can maintain the pressure is needed and the facility cost can be lowered.

### Method for Producing Saccharides Containing Glucose as Main Component

A method for producing saccharides containing glucose as a main component according to this embodiment includes degrading, by using enzymes that degrade cellulose and/or hemicellulose, cellulose and/or hemicellulose contained in a biomass pretreated by the biomass pretreatment method of this embodiment so as to obtain saccharides containing glucose as a main component.

According to the method for producing saccharides containing glucose as a main component, a biomass pretreated by the biomass pretreatment method of the embodiment is prepared first.

Next, the biomass and an aqueous solution (aqueous enzyme solution) containing an appropriate amount of cellulase suitable for degrading cellulose and/or hemicellulose in the biomass are placed in a reactor (enzymatic degradation tank), and the biomass and the aqueous enzyme solution are mixed (preparation step).

In the preparation step, the pH of the reactor solution is adjusted so that the pH condition of the reactor solution is optimum for the enzymes used. The temperature of the reactor is also adjusted so that the temperature conditions are optimum for the enzymes used.

In the preparation step, the pH of the mixture of the biomass, the aqueous enzyme solution, and additives is preferably adjusted so that the enzymes can actively function. Specifically, the pH of the reaction-system aqueous solution is preferably adjusted to 4 to 6.

Moreover, in the preparation step, the temperature of the mixture is preferably adjusted so that the enzymes can actively function. Specifically, the temperature of the reaction system is preferably increased to 40° C. to 60° C.

The biomass concentration in the reactor is preferably 5 g to 50 g relative to 100 mL of the solution, namely, is preferably 5 w/v % to 50 w/v %, and more preferably 10 g to 30 g relative to 100 mL of the solution, namely, 10 w/v % to 30 w/v %.

Cellulase is used as the enzymes for degrading the biomass. In the case where a large amount of hemicellulose is contained in the biomass, xylanase and mannanase that degrade hemicellulose are preferably added in addition to cellulase.

The mixture is stirred by using a stirring blade or the like.

In this embodiment, the mixture is stirred and mixed gently in the reactor so that the enzymes contained in the aqueous enzyme solution are not excessively deactivated; as a result, the biomass (cellulose and/or hemicellulose) can be efficiently enzymatically saccharified.

In this enzymatic saccharification reaction step, the temperature of the mixture is preferably adjusted so that the enzymes function actively. Specifically, the temperature is preferably retained at 40° C. to 60° C.

The enzymatic saccharification reaction step is continued until enzymatic saccharification of the biomass has fully progressed to a point that the reaction will no longer proceed. For example, the enzymatic degradation of the biomass is performed at 40° C. to 60° C. for about 2 to 20 days.

In a method for producing saccharides containing glucose as a main component according to this embodiment, a biomass pretreated by the biomass pretreatment method of this embodiment is supplied; thus, enzymes that hydrolyze cellulose and/or hemicellulose do not come into contact with components, such as organic acids, that inhibit enzymatic saccharification and the area in which the enzymes contact the cellulose and/or hemicellulose can be increased. As a result, the reaction rate can be improved and the amount of enzymes used can be reduced.

### EXAMPLES

The present invention will now be described in further detail by using Examples and Comparative Examples; however, the present invention is not limited to the Examples below.

### Example 1

Eucalyptus was hydrothermally exploded and the steam-exploded eucalyptus was boiled in hot water of 100° C. for 5 minutes.

Then the eucalyptus was degraded with cellulase under the following conditions:

Weight of eucalyptus: 10 g/dry

Amount of cellulase added: 20 mg/protein

Amount of solution: 50 mL

Temperature: 50° C.

pH: 5

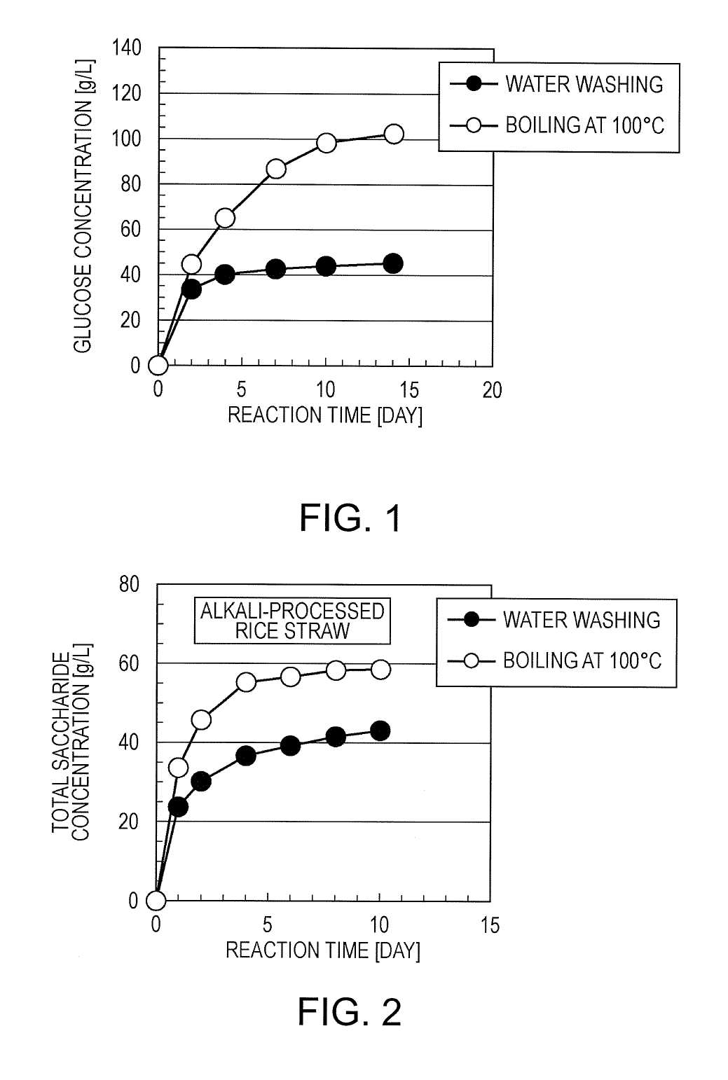

The relationship between the enzymatic degradation reaction time (min) and the concentration of glucose obtained (g/L) was investigated. The results are indicated in FIG. 1.

### Comparative Example 1

Steam-exploded eucalyptus was washed with 20° C. water for 5 minutes.

Then the eucalyptus was degraded with cellulase as in Example 1.

The relationship between the enzymatic degradation reaction time (min) and the concentration of glucose obtained (g/L) was investigated. The results are indicated in FIG. 1.

The results of FIG. 1 show that the concentration of glucose obtained by enzymatic degradation is significantly improved in Example 1 in which steam-exploded eucalyptus was boiled in 100° C. hot water compared to Comparative Example 1 in which the steam-exploded eucalyptus was simply washed with 20° C. water.

### Example 2

Rice straw was pretreated with an aqueous sodium hydroxide solution and boiled in 100° C. hot water for 5 minutes.

Then the rice straw was degraded with cellulase under the following conditions:

Weight of rice straw: 10 g/dry

Amount of cellulase added: 20 mg/protein

Amount of solution: 50 mL

Temperature: 50° C.

pH: 5

The relationship between the enzymatic degradation reaction time (min) and the concentration of glucose obtained (g/L) was investigated. The results are indicated in FIG. 2.

### Comparative Example 2

Rice straw pretreated with an aqueous sodium hydroxide solution was washed with 20° C. water for 5 minutes.

Then the rice straw was degraded with cellulase as in Example 2.

The relationship between the enzymatic degradation reaction time (min) and the concentration of glucose obtained (g/L) was investigated. The results are indicated in FIG. 2.

The results in FIG. 2 show that the concentration of glucose obtained by enzymatic degradation is significantly improved in Example 2 in which rice straw pretreated with an aqueous sodium hydroxide solution was boiled in 100° C. hot water compared to Comparative Example 2 in which rice straw pretreated with an aqueous sodium hydroxide solution was simply washed with 20° C. water.

### Example 3

Rice straw was pretreated with a diluted aqueous sulfuric acid solution and boiled in 100° C. hot water for 5 minutes.

Then the rice straw was degraded with cellulase under the following conditions:

Weight of rice straw: 10 g/dry

Amount of cellulase added: 20 mg/protein

Amount of solution: 50 mL

Temperature: 50° C.

pH: 5

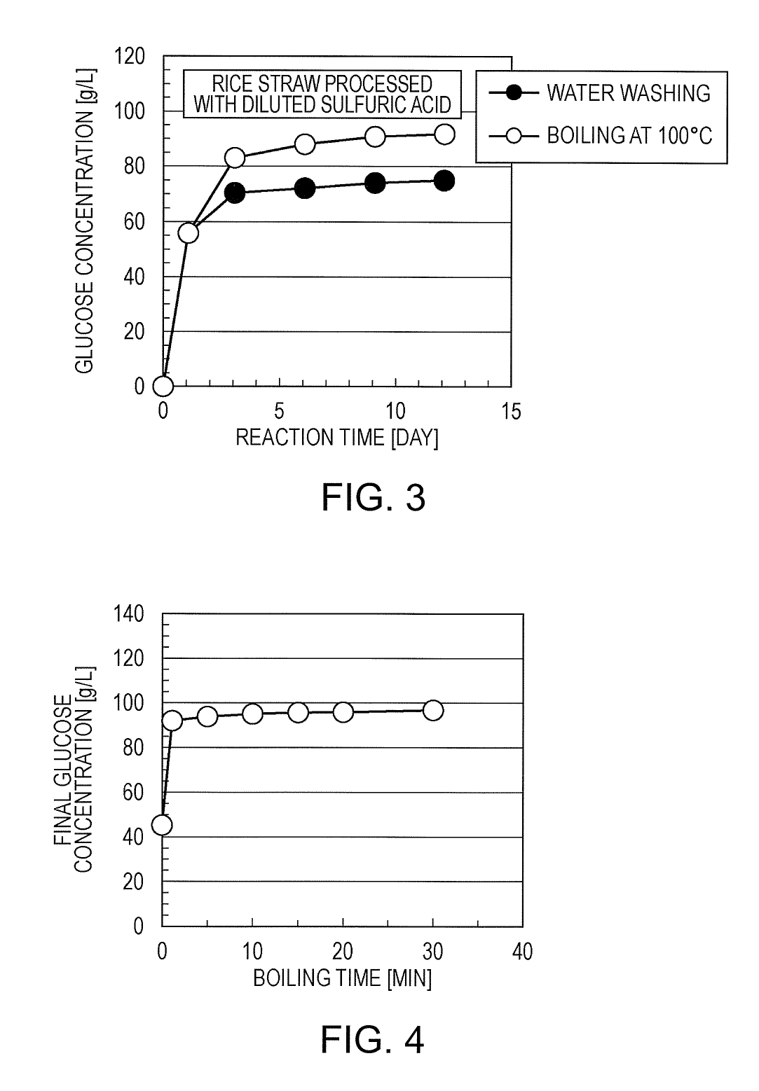

The relationship between the enzymatic degradation reaction time (min) and the concentration of glucose obtained (g/L) was investigated. The results are indicated in FIG. 3.

### Comparative Example 3

Rice straw pretreated with a diluted aqueous sulfuric acid solution was washed with 20° C. water for 5 minutes.

Then the rice straw was degraded with cellulase as in Example 3.

The relationship between the enzymatic degradation reaction time (min) and the concentration of glucose obtained (g/L) was investigated. The results are indicated in FIG. 3.

The results in FIG. 3 show that the concentration of glucose obtained by enzymatic degradation is significantly improved in Example 3 in which rice straw pretreated with a diluted aqueous sulfuric acid solution was boiled in 100° C. hot water compared to Comparative Example 3 in which rice straw pretreated with a diluted aqueous sulfuric acid solution was simply washed with 20° C. water.

### Experimental Example

Bagasse was steam-exploded and the steam-exploded bagasse was boiled in 100° C. hot water.

The length of time bagasse was boiled was varied such as 1 minute, 5 minutes, 10 minutes, 15 minutes, 20 minutes, and 30 minutes.

The bagasse was then degraded by cellulase under the following conditions:

Weight of bagasse: 10 g/dry

Amount of cellulase added: 20 mg/protein

Amount of solution: 50 mL

Temperature: 50° C.

pH: 5

Length of time of enzymatic degradation reaction: 15 days

The relationship between the length of time steam-exploded bagasse was boiled and the concentration of glucose obtained (g/L) was investigated. The results are indicated in FIG. 4.

The results in FIG. 4 show that the concentration of glucose finally obtained becomes substantially constant when the length of time the steam-exploded bagasse is boiled is 1 to 10 minutes. Accordingly, it is assumed that the length of time bagasse is boiled is preferably about 1 to 10 minutes.

### INDUSTRIAL APPLICABILITY

Provided are a biomass pretreatment method that enables high-yield production of saccharides containing glucose as a main component from cellulose and/or hemicellulose contained in a biomass by using less enzymes, and a method for manufacturing saccharides containing glucose as a main component using the biomass pretreatment method.

1. A method for pretreating a biomass to be supplied to a method for producing saccharides containing glucose as a main component, the method for producing saccharides including degrading cellulose and/or hemicellulose contained in a biomass with enzymes that degrade cellulose and/or hemicellulose, comprising:
a first process performed on the biomass so as to improve contact efficiency between the cellulose and/or hemicellulose and the enzymes that degrade the cellulose and/or hemicellulose; and
a second process of removing a substance containing at least an organic acid from the biomass subjected to the first process, the second process including placing the biomass subjected to the first process into a container that has a vent communicating with outside and heating the biomass subjected to the first process at a temperature equal to or lower than a boiling temperature of water by using water while leaving the vent constantly open.

2. The method for pretreating a biomass according to claim 1, wherein the first process is a steam explosion process or a diluted sulfuric acid process.

3. The method for pretreating a biomass according to claim 1, wherein the second process involves heating the biomass by using water at 100° C. or lower.

4. The method for pretreating a biomass according to claim 3, wherein in the second process, X representing a temperature of water used to heat the biomass and Y representing a length of time the biomass is heated, and X and Y satisfy formula (1) below:
Y≧0.0175X²−3.485X+174 (1)

5. A method for producing saccharides containing glucose as a main component, comprising degrading, by using enzymes that degrade cellulose and/or hemicellulose, cellulose and/or hemicellulose contained in a biomass pretreated by the method for pretreating a biomass according to claim 1 so as to obtain saccharides containing glucose as a main component.

6. A method for producing saccharides containing glucose as a main component, comprising degrading, by using enzymes that degrade cellulose and/or hemicellulose, cellulose and/or hemicellulose contained in a biomass pretreated by the method for pretreating a biomass according to claims 2 so as to obtain saccharides containing glucose as a main component.

7. A method for producing saccharides containing glucose as a main component, comprising degrading, by using enzymes that degrade cellulose and/or hemicellulose, cellulose and/or hemicellulose contained in a biomass pretreated by the method for pretreating a biomass according to claims 3 so as to obtain saccharides containing glucose as a main component.

8. A method for producing saccharides containing glucose as a main component, comprising degrading, by using enzymes that degrade cellulose and/or hemicellulose, cellulose and/or hemicellulose contained in a biomass pretreated by the method for pretreating a biomass according to claims 4 so as to obtain saccharides containing glucose as a main component.
